(19) United States
(12) Patent Application Publication
(10) Pub. No.: US 2024/0307519 A1
(43) Pub. Date: Sep. 19, 2024

(54) MICROENCAPSULATED STERNE VACCINE

(71) Applicant: THE TEXAS A&M UNIVERSITY SYSTEM, College Station, TX (US)

(72) Inventors: Jamie Suzanne Benn, Madison, WI (US); Chase M. Nunez, Caldwell, TX (US); Walter E. Cook, Milano, TX (US); Allison Ficht, College Station, TX (US); Thomas A. Ficht, College Station, TX (US); Sankar P. Chaki, Bryan, TX (US)

(21) Appl. No.: 18/576,813

(22) PCT Filed: Jul. 7, 2022
(86) PCT No.: PCT/US2022/036329

§ 371 Date: Jan. 5, 2024

Related U.S. Application Data
(60) Provisional application No. 63/219,135, filed on Jul. 7, 2021.

(51) Int. Cl. A61K 39/07; A61K 39/00; C12N 11/10; C12R 1/07

(52) U.S. Cl. CPC A61K 39/07; C12N 11/10; A61K 2039/542; A61K 2039/552; A61K 2039/55555; A61K 2039/575; C12R 2001/07

(57) ABSTRACT

Methods and compositions for the immunization of animals and humans using an immunization or vaccine that comprises B. anthracis Sterne strain 34F2 spores suspended in alginate in an amount sufficient to protect an animal or human from a lethal dose of anthrax.

### TECHNICAL FIELD OF THE INVENTION

The present invention relates in general to methods and compositions for the treatment of Bacillus sp.-induced diseases, and more particularly, to a method of making a novel oral vaccine against Bacillus anthracis.

### STATEMENT OF FEDERALLY FUNDED RESEARCH

Not applicable.

### REFERENCE TO A SEQUENCE LISTING

Not applicable.

### BACKGROUND OF THE INVENTION

Without limiting the scope of the invention, its background is described in connection with vaccines.

Anthrax infections have plagued humans and animals alike for millennia, possibly even causing the fifth and sixth plagues of Egypt.¹ The causative agent, Bacillus anthracis, has been studied since the beginning of microbiology but even after more than a century of scientific studies, the anthrax vaccination field has made little progress, especially a veterinary anthrax vaccine.^{1,2} Consolidated data from the last twenty years found a worldwide distribution with reports of the disease on every habitable continent, yet most animals remain unvaccinated.³ While it may be prudent to mention that the incidence of human infection can be decreased with adequate livestock and wildlife vaccination policies, it should also be of great concern that free-ranging livestock and wildlife populations worldwide are unprotected against anthrax outbreaks that can cause catastrophic harm to sensitive wildlife conservation efforts.³⁻⁶

The current veterinary vaccine, historically referred to as the Sterne vaccine, uses B. anthracis Sterne strain 34F2 spores (Sterne spores) that have naturally lost the pXO2 plasmid and therefore can no longer produce the poly-γ-D-glutamic acid capsule, also known as the anti-phagocytic capsule.⁶ The original formulation of the Sterne vaccine, which is still in use today, consists of Sterne spores suspended in saponin and has been used to vaccinate domesticated livestock against anthrax since its discovery in the late 1930's.^{1,7} Despite decades of successful protections, the Sterne vaccine is outdated, impractical, known to vary in its potency and can cause adverse reactions, occasionally even death.⁸ The Sterne vaccine is administered as a subcutaneous injection, which is a highly impractical method of vaccination for free-ranging livestock and wildlife.¹ Without a reasonable method of wildlife vaccination, yearly anthrax outbreaks in national parks and other wildlife areas worldwide pose economic, ecological and conservational burdens to wildlife health professionals.^{3,7,9,10} Even with these yearly outbreaks, the anthrax spore distribution in these areas is undetermined so it isn't possible to vaccinate wildlife based on an estimated risk of exposure.¹¹ The most feasible way to protect wildlife in these areas would be via oral vaccination however, after results from a previous study demonstrated that the Sterne vaccine is incapable of eliciting an immune response following oral vaccination, the urgent need for an effective oral anthrax vaccine for wildlife has never been more evident.¹²

Other research groups in the oral anthrax vaccination field have reported encouraging results from vaccines expressing a recombinant form of anthrax protective antigen in a variety of bacterial, viral or plant-based expression systems.¹³⁻¹⁶ Unfortunately, exposure to a single recombinant antigen may not stimulate sufficient immune activity to protect against fully virulent exposure. Studies have demonstrated that immunizing mice and guinea pigs with inactivated anthrax spores and recombinant antigens elicited enhanced protection against B. anthracis suggesting that anthrax spore associated antigens are also important for vaccine induced protection.^{17,18} However, inactivated spores and recombinant antigens remain less protective than live-attenuated vaccines.¹⁷

Despite these improvements, what is needed is an improved immunization that protects against anthrax outbreaks that can cause catastrophic harm to sensitive wildlife conservation efforts.

### SUMMARY OF THE INVENTION

The present invention relates to methods and compositions for the treatment of Bacillus-induced diseases and disorders. In preferred embodiments, the invention relates to vaccines.

As embodied and broadly described herein, an aspect of the present disclosure relates to an immunization against Bacillus anthracis comprising: B. anthracis Sterne strain 34F2 spores suspended in an alginate in an amount sufficient to protect an animal or human from a lethal dose of anthrax. In one aspect, the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the oral immunization further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5. 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

As embodied and broadly described herein, an aspect of the present disclosure relates to a vaccine comprising: B. anthracis Sterne strain 34F2 spores suspended in an alginate, wherein the spores are provided in an amount sufficient to protect an animal or human from a lethal dose of anthrax. In one aspect, the immunization is for oral administration further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the vaccine further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 02.5, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

As embodied and broadly described herein, an aspect of the present disclosure relates to a method for prophylaxis, amelioration of symptoms, or any combinations thereof against Bacillus anthracis in a human or animal subject comprising the steps of: identifying the human or animal subject in need of the prophylaxis, amelioration of symptoms, or any combinations thereof against Bacillus anthracis; and administering a therapeutically effective amount of an attenuated immunization against Bacillus anthracis comprising: B. anthracis Sterne strain 34F2 spores suspended in an alginate, wherein the immunization is provided in an amount sufficient to protect an animal or human from a lethal dose of anthrax. In one aspect, the immunization is an oral immunization and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the immunization further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 2, 2.5, 3, 4, 5, 6, 7, 8, 9, 10% weigh to volume (w/v).

As embodied and broadly described herein, an aspect of the present disclosure relates to a method of making an attenuated vaccine against anthrax (Bacillus anthracis) comprising: suspending B. anthracis Sterne strain 34F2 spores in alginate, wherein the alginate neutralizes positively charged amino acids, wherein the amount of the vaccine is sufficient to protect an animal or human from a lethal dose of anthrax. In one aspect, the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the vaccine further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 2, 2.5, 3, 4, 5, 6, 7, 8, 9, 10% weigh to volume (w/v).

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows uncoated microcapsule diameter changes to simulated gastrointestinal environments. Uncoated microcapsules were suspended in simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions. The capsule diameters after exposure to simulated gastrointestinal fluids were observed in brightfield and measured in ImageJ. Data is reported as the average capsule diameter for the group in μm±the standard deviation. Data for coated microcapsules is not shown.

FIG. 2 shows uncoated and coated microcapsule responses to simulated gastrointestinal environments. Representative brightfield images of microcapsule samples following exposure to simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions.

FIG. 3 is an illustration of the uncoated microcapsules created in this study. Created with BioRender.com.

FIG. 4 is a graph that shows the release patterns of different microcapsule formulations with and without the protein shell in simulated ruminant digestive environments.

FIG. 5 is a graph that shows microcapsule diameter changes for different microcapsule formulations to simulated ruminant digestive environments for the estimated time that capsules would spend in each digestive organ.

FIG. 6 is a graph that shows IgG responses in white-tailed deer (Odocoileus virginianus) following subcutaneous and intramuscular vaccination with the Sterne vaccine or oral vaccination with uncoated capsules, PLL capsules and PLL/VpB capsules at different doses.

FIG. 7 is a graph that shows in vitro toxin neutralizing abilities of antibodies from subcutaneous administered Sterne Vaccine and orally administered uncoated capsules, PLL capsules and PLL/VpB capsules at different doses in white-tailed deer.

FIG. 8 is a graph that shows IgG responses in axis deer (Axis axis) following subcutaneous and intramuscular vaccination with the Sterne vaccine or oral vaccination with uncoated capsules.

FIG. 9 is a graph that shows in vitro toxin neutralizing abilities of antibodies from subcutaneous or intramuscularly injected Sterne Vaccine and 6 mL of orally administered uncoated capsules in axis deer.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

An oral vaccine against anthrax (Bacillus anthracis) is urgently needed to prevent annual anthrax outbreaks that are causing catastrophic losses in free-ranging livestock and wildlife worldwide. The Sterne vaccine, the current injectable livestock vaccine, is a suspension of live attenuated B. anthracis Sterne strain 34F2 spores (Sterne spores) in saponin. It is not effective when administered orally and individual subcutaneous injections are not a practical method of vaccination for wildlife.

The present invention is the development of a microencapsulated oral vaccine against anthrax. Evaluating Sterne spore stability at varying pH's in vitro revealed that spore exposure to pH 2 results in spore death, confirming that protection from the gastric environment is of main concern when producing an oral vaccine. Therefore, Sterne spores were encapsulated in alginate.

The present inventors found that an effective vaccine can be made without the need for an additional protein coating.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

As used herein, the term “subject” as used herein refers to any mammal, preferably livestock, wildlife, a human patient, or domestic pet. It is intended that the term “subject” encompass both human and non-human mammals, including, but not limited to cervids, bovines, caprines, ovines, equines, porcines, felines, canines, wild-game, such as deer, buffalo, etc., and other wildlife, as well as humans.

In preferred embodiments, the “subject” is a cervid (e.g., a deer) or a human and it is not intended that the present invention be limited to these groups of animals.

As used herein the term “immunogenically-effective amount” refers to that amount of an immunogen required to generate an immune response (e.g., invoke a cellular response and/or the production of protective levels of antibodies in a host upon vaccination).

In a specific embodiment, the term “pharmaceutically acceptable” means approved by a regulatory agency of the federal or a state government or listed in the U.S. Pharmacopeia or other generally recognized pharmacopeia for use in animals and in humans.

As used herein, “vitelline protein B” or “VpB” refers to a non-immunogenic, proteolysis resistant protein from Fasciola hepatica. Recombinant vitelline protein B (VpB) can be obtained as follows: ˜25 ng of Fasciola hepatica genomic DNA is isolated and polymerase chain reaction (PCR) used to amplify the coding region of VpB. The amplified Fasciola hepatica genomic DNA was then cloned into an expression vector to ectopically express VpB in E. coli bacteria. E. coli bacterial stock is stored at −80. Finally, to make the recombinant VpB, the E. coli grown and the recombinant VpB is isolated and purified. The making, isolation and purification of recombinant VpB is taught by the present inventors, in at least, U.S. Patent Publication Nos. 20050260258, 20120156287, and 20170135958, relevant portions incorporated herein by reference.

As used herein, the term “carrier” as used herein refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The present invention relates to methods and compositions for the treatment of Bacillus induced diseases and disorders. In preferred embodiments, the invention relates to vaccines. In additional embodiments, the invention relates to formulations capable of releasing said live vaccines at a controlled rate of release in vivo. In further embodiments, the invention relates to an oral B. anthracis Sterne strain 34F2 vaccine.

As previously mentioned, no federally approved or commercially available oral veterinary anthrax vaccines are available anywhere worldwide; there simply are no currently known or published existing vaccine alternatives to prevent annual anthrax outbreaks that are causing catastrophic losses in free-ranging livestock and wildlife worldwide. Additionally, the currently approved human anthrax vaccines in the United States and internationally require intramuscular injections with up to five doses to initiate immunity, followed by yearly injections to prolong immunity.

The present invention provides for an attenuated, live B. anthracis Sterne strain 34F2 spore vaccine for oral administration coated with alginate. Drug delivery materials have historically been derived from many sources including commodity plastics and textile industries and have been incorporated into vehicles as diverse as pH responsive hydrogels and polymer microparticles or implants designed for surface or bulk erosion as disclosed in Langer RaP, N. A. (2003) Bioengineering, Food and Natural Products 49, 2990-3006, incorporated herein by reference. In the case of controlled release formulations, a drug is typically released by diffusion, erosion or solvent activation and transport. In most cases, the desired polymer characteristics include biocompatibility, lack of immunogenicity, capability of breakdown by the body and water solubility. Many of the processes used to entrap pharmaceuticals involve harsh organic solvents which are bacteriocidal and capable of denaturing proteins. When considering controlled release vehicles for the entrapment of active enzymes or living cells, new alternatives are needed. A number of milder processes based on established technologies and variations have recently been applied to the delivery of active protein agents such as insulin, erythropoietins and chemokines as provided for in Marschutz et al. (2000) Biomaterials 21, 1499-07. Takenaga et al. (2002) J Control Release 79, 81-91. and Qiu et al. (2003) Biomaterials 24, 11-18., all of which are incorporated by reference, or as encapsulants for living cells to permit transplantation as disclosed in Young et al. (2002) Biomaterials 23, 3495-3501, hereby incorporated by reference.

As used herein, the term “alginate” refers generically to a naturally occurring biopolymer, is especially well suited to the entrapment of living cells. One type of alginate is a linear unbranched polysaccharide composed of 1-4′-linked β-D-mannuronic acid and α-L-guluronic acids in varying quantities. Generally, alginate is made from alginic acid, also referred to as algin, which is a polysaccharides made from the cell walls of brown algae that is hydrophilic and forms a viscous gum when hydrated. All such types of alginate and their salts, e.g., sodium, potassium and calcium, can be used with the present invention. Alginate polymers are highly water-soluble and easily crosslinked using divalent cations such as Ca²⁺ or polycations such as poly-L-lysine (PLL) as provided for in Wee & Gombotz (1998) Adv Drug Deliv Rev 31, 267-285, relevant portions hereby incorporated by reference. The relatively mild conditions required to produce either an alginate matrix or particle is compatible with cell viability. Entrapment in alginate has been shown to greatly enhance viability and storage as provided for in Cui et al (2000) Int J Pharm 210, 51-59 and Kwok et al. (1989) Proc. Int. Symp. Contol. Release Bioact. Mater. 16, 170-171, both of which are incorporated by reference. The physical properties such as porosity, rate of erosion, and release properties may be modulated through mixing alginates of different guluronic acid composition and through applying different coatings to the matrix as provided for in Wee & Gombotz (1998) Adv Drug Deliv Rev 31, 267-285. While in no way limiting the scope of the present invention, it is generally thought that release of a biomolecule from alginate matrices generally occurs through i) diffusion through pores of the polymer or ii) erosion of the polymer network. In general, the alginate matrix is stabilized under acidic conditions, but erodes slowly at pH of 6.8 or above.

Pharmaceutical Formulations: The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). In one embodiment, the vaccine is encapsulated using materials described in U.S. Patent Application Publications No. 2005/0260258, 2012/0156287, and 2017/0135958, relevant portions hereby incorporated by reference.

In a preferred embodiment, the active compound and optionally another therapeutic or prophylactic agent are formulated in accordance with routine procedures as pharmaceutical compositions adapted for administration to animals or human beings. Typically, the active compounds for administration are solutions in sterile isotonic aqueous buffer. Where necessary, the compositions can also include a solubilizing agent. Compositions for administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of feed, bait, tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an oral administration of the active compound. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the active compound can be delayed or prolonged by proper formulation. For example, a slowly soluble pellet of the active compound can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long acting, by dissolving or suspending the compound in oily or emulsified vehicles, which allow it to disperse only slowly in the serum.

Compositions for use in accordance with the present invention can be formulated in conventional manner using one or more physiologically acceptable carriers or excipients. Thus, the compound and optionally another therapeutic or prophylactic agent and their physiologically acceptable salts and solvates can be formulated into pharmaceutical compositions for administration by oral (typically feed/bait or in a liquid) or mucosal (such as buccal or sublingual) administration.

For widespread or herd immunization, the present invention can be provided in bait. The bait can be a generic bait made from, e.g., pellets, hay, grasses, common baiting materials, etc. Generally, the livestock bait will be suitable for use by any species of any age or size, including but not limited to cattle, sheep, goats, horses, mules, donkeys, bison, alpacas, llamas, deer, elk, exotic animals, zoo animals, game animals, and wildlife animals.

For oral administration, the compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for administration can be suitably formulated to give controlled release of the active compound. The microencapsulated vaccine gives a controlled release or continual boosting effect.

For buccal administration the compositions can take the form of feed, pellets, tablets or lozenges formulated in conventional manner.

In addition to the formulations described previously, the compositions can also be formulated as a depot preparation. Such long-acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration. In certain embodiments, the pack or dispenser contains one or more unit dosage forms containing no more than the recommended dosage formulation as determined in the Physician's Desk Reference (62nd ed. 2008, herein incorporated by reference in its entirety).

The active compound and optionally other prophylactic or therapeutic agents can also be administered by infusion or bolus injection and can be administered together with other biologically active agents. Administration can be local or systemic. The active compound and optionally the prophylactic or therapeutic agent and their physiologically acceptable salts and solvates can also be administered by inhalation or insufflation (either through the mouth or the nose). In a preferred embodiment, local or systemic parenteral administration is used.

The present invention uses alginate encapsulation.¹⁹⁻²¹ Alginate is naturally indigestible in mammalian systems which can be implemented as a natural controlled release vehicle.^{22,23} Additionally, the mild gelation conditions permit entrapment of the desired capsule load without significantly affecting the viability.²² Post-gelation, the viability of the capsule load is maintained by stability of the microcapsule, particularly in gastric environments which has proven overwhelmingly beneficial for the development of probiotics.²⁰ Alginate has also demonstrated bio-adhesive properties when interacting with mucosal tissues. Combined with the depot effect of alginate capsules, these bio-adhesive properties ensure that the capsule load is repeatedly released in close proximity to target cells.¹⁹

The novel formulation of the present invention showed the stability of microcapsules as enteric delivery vehicles. The inventors also demonstrated the immunogenicity of microencapsulated Sterne spores and observed a pronounced increase in the resulting antibody response from oral vaccination showing that microencapsulated of Sterne spores are an alternative anthrax vaccine formulation capable of efficient and protective vaccination of free-ranging livestock and wildlife.

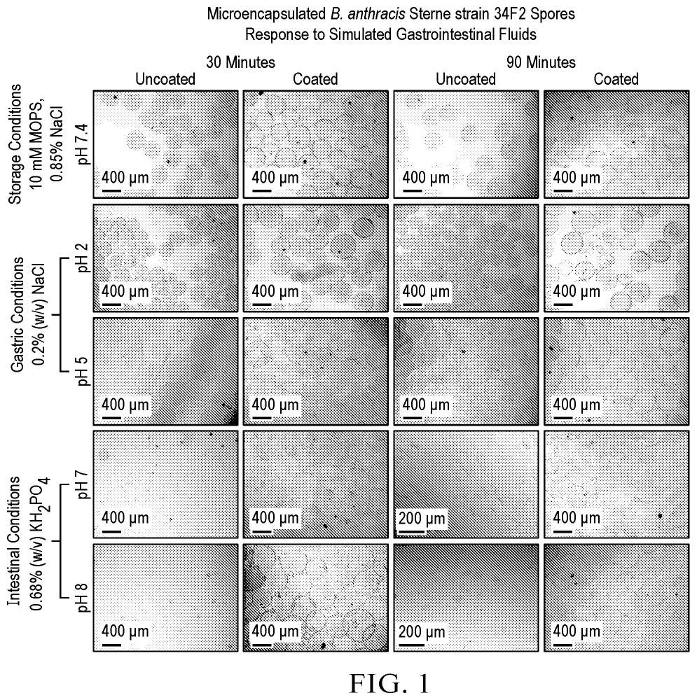

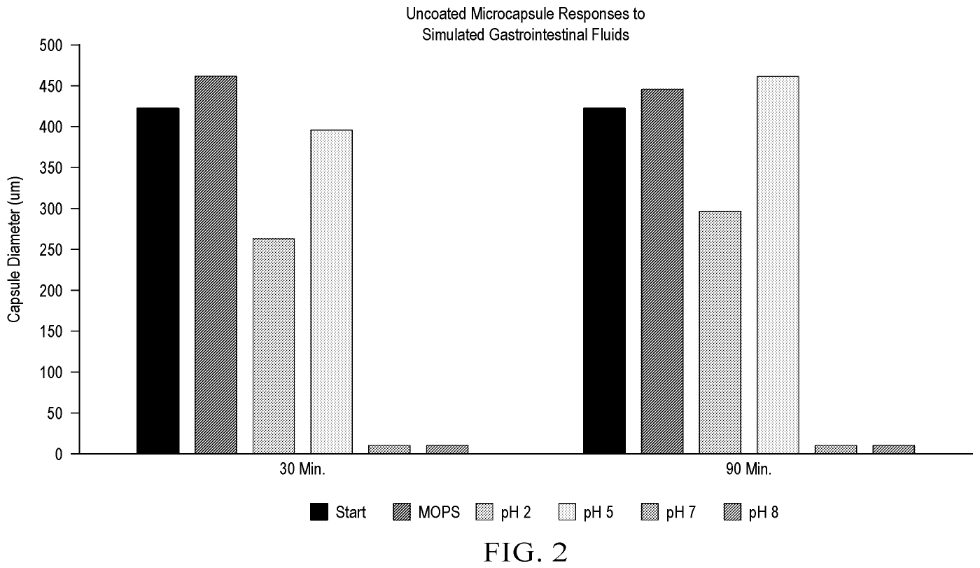

Comparison of microcapsule formulations in gastrointestinal environments. Microcapsules were exposed to GI fluids to observe the relative stability in simulated gastrointestinal conditions.³³ Microcapsule samples were suspended in MOPS buffer as a negative control and simulated GI fluids at pH 2, 5, 7 and 8 for 30 and 90 minutes at 37° C. with shaking. At pH 2, uncoated capsules were shown to decrease in diameter compared to neutral storage conditions in MOPS, whereas at pH 5 uncoated capsules experienced significant swelling (FIG. 1). At neutral pHs, uncoated capsules disintegrated. (FIG. 1). These patterns were also observed in uncoated capsules after 90 minutes in GI fluids, simply to a higher degree as a result of the extended exposure. In comparison, coated capsules exhibited overall stability in all GI fluids by preventing shrinking at pH2 and complete capsule dissolution at pH 7 and 8 (FIG. 2).

Microcapsule vaccines induce anthrax specific antibody responses. Antibody levels against anthrax protective antigen were measured by end-point dilution ELISAs at 14 days post vaccination. All vaccines elicited antibody responses with the subcutaneously injected Sterne Vaccine exhibiting the strongest. Of the orally inoculated vaccines, white-tailed deer developed the strongest antibody response to the Uncoated-Large Dose, followed by the Coated-Large Dose, then the Uncoated-Small Dose.

The benefits of oral vaccine delivery cannot be overstated, particularly when it comes to protecting free-ranging livestock and wildlife from current and emerging infectious diseases such as anthrax. Development of oral vaccines can allow for easy, wide-spread vaccination policies without needing to deal with the labor-intensive programs and painful injections associated with the majority of today's human and animal vaccines. It is also possible that effective oral vaccines may be intrinsically more stable and have longer shelf-lives as a collateral benefit of the stability required for transit through the gastrointestinal tract. Furthermore, oral vaccines can lead to enhanced efficacy with less adverse effects due to mucosal immunity and oral delivery.

For all of these reasons and more, an alternative anthrax vaccine formulation specifically for oral administration is urgently needed to protect animals worldwide from potentially catastrophic anthrax outbreaks.^{3,12} Many wildlife health professionals have demanded a new veterinary anthrax vaccine because individual hand-injections for each and every animal is not a practical method of vaccination for wildlife and a recent study demonstrated that oral vaccination with the Sterne vaccine is not effective.^{1,12} Also, sustained protection from the Sterne vaccine can only be achieved with yearly boosters which requires a yearly cycle of troublesome injections with the potential for adverse reactions.¹ To resolve the many issues associated with anthrax outbreaks and vaccination, the inventors developed and evaluated a novel anthrax vaccine formulation for oral vaccination. Results of the inventors' study demonstrate that oral vaccination with uncoated microencapsulated B. anthracis Sterne strain 34F2 spores can induce antibody production in the cervid model.

Oral vaccination is a common goal throughout the entire vaccinology field but there are still a limited number of oral vaccines approved for animal and human use because the main obstacle facing oral vaccination is, ironically, oral vaccination itself.³⁴⁻³⁶ The principle of oral vaccination is completely dependent on getting sensitive antigens through the harsh, gastric environment that was evolutionarily designed specifically to prevent that exact thing from happening. In contrast, gastrointestinal pathogens, such as anthrax, have also evolved over thousands of years to survive the gastric environment for eventual uptake in the small intestine but these pathogen survival strategies are not typically conserved in live attenuated organisms, which is a reliable vaccine format. Such is the case with B. anthracis Sterne strain 34F2. Upon exposure to a simulated gastric environment, there was a severe decrease in the viable Sterne spore titer. This shows that development of an oral vaccine with the Sterne strain must involve some protection to ensure passage through the stomach. Given that the majority of anthrax infections in wildlife are gastrointestinal, it can be reasoned that fully virulent anthrax spores are able to survive passage through a harsh acidic environment to establish infections following uptake in the small intestine. In comparison to the experiments performed here with the pXO2-negative Sterne strain, this shows that fully virulent anthrax spores may be better equipped to survive the gastrointestinal environment due to retention of the pXO2 plasmid. Alginate encapsulation was able to shield Sterne spores enough through the gastric environment to induce an immune response following oral vaccination.

First, the stabilizing and shielding abilities of the microcapsules produced in this study was assessed by observing the microcapsule responses to simulated gastrointestinal environments. When alginate capsules are formed in a cross-linking solution, guluronate residues in the alginate cooperatively bind Ca²⁺ ions from the solution, thus cross-linking the alginate polymers to the “pre-gel” state.^{21,24} Exposure of a calcium cross-linked pre-gel to nongelling cations, such as Na⁺, will reduce the mechanical stability of the alginate gel and possibly disintegrate the entire polymer matrix.^{21,25} This can be prevented by adding additional cross-linked layers to the microcapsules, thus resulting in more stable capsules which the inventors have demonstrated here by exposing coated and uncoated microcapsules to gastrointestinal environments.³⁷

The added stability of these layers can be assessed through changes in microcapsule shrinking, swelling and overall morphology. Changes in the alginate polymer network such as these can greatly affect the rate of diffusion through and the erosion of the network, thereby altering the antigen release rate.^{22,38,39}

The present invention shows improvement on the prior art by eliminating the need for any protein shell. A second challenge to oral vaccination, after having endured the harsh gastric environment, is to ensure antigen transport across the intestinal epithelia followed by antigen-presenting cell activation.

The findings of this study exemplify the advantages and efficacy of Sterne spore microencapsulation. It is further demonstrated that the protein shell was not essential for maintaining the controlled release aspects of alginate microcapsules. Following a single vaccination dose in deer, microencapsulated Sterne spores generated a significant antibody response via oral vaccination. This immune response can be further enhanced by inoculating a higher bacterial dose with limited adverse effects.

While the results presented here reveal the great potential for this oral vaccine formulation, the majority of wildlife species affected by anthrax are ruminants and thus present further challenges to oral vaccination in the form of three additional stomachs and rumination.⁵³ Continued research is essential to optimize this vaccine for ruminant species. Future work will involve in vivo studies in a ruminant model to evaluate effective oral vaccination doses and the effect of vaccine boosters. It will also be critical to do an in vivo challenge experiment in a ruminant model to fully demonstrate the protective efficacy of this vaccine. The vaccine can be added in a wildlife bait to establish a practical wildlife vaccination method against anthrax.

In summary, the present invention is the first effective oral vaccination against anthrax. It is demonstrated herein, for the first time, the generation of protective antibody responses from oral vaccination with B. anthracis Sterne strain 34F2 spores without the need for a protein coating, which can be adapted such that the Sterne spore is effective for oral vaccination of free-ranging livestock and wildlife.

Preparation of Sterne spores. All bacteria used in this experiment were cultured from a vial of the Anthrax Spore Vaccine (Sterne Vaccine) from Colorado Serum Company (Denver, CO, USA), the North American commercial producer of the Sterne vaccine. The Sterne Vaccine consists of live attenuated B. anthracis Sterne strain 34F2 spores in saponin were used to inoculate Luria Broth (LB) and cultured in liquid form at 37° C. with shaking for 5-7 days, or until sporulated. Spores were harvested by centrifugation and washed repeatedly with sterile water or 2.5 mM D-alanine or 5 mM D-alanine. The skilled artisan will understand that the amount of D-alanine can be modulated to prevent spore germination, such as 0.1, 0.2, 0.3, 0.4, 0.5. 0.75, 1, 2, 2.5, 3, 4, 5, 6, 7, 8, 9, 10 or more mM. Remaining vegetative cells were killed by heating at 68° C. for 1 hour and removed by filtering through a 3.1 m filter, if needed, resulting in a suspension of pure Sterne spores. The final Sterne spore concentration was estimated by plating serial dilutions on LB agar.

Vaccine preparation. Sterne vaccine. The Sterne Vaccine is distributed by Colorado Serum with a recommended 1 ml dose of between 4×10⁶ and 6×10⁶ viable Sterne spores in saponin for use in cattle, sheep, goats, swine and horses.¹² This dosage range was simplified to 5×10⁶ spores/ml for the purposes of this experiment and was used exactly as received from Colorado Serum Company. Identical booster doses are recommended two weeks post-vaccination in highly contaminated areas.

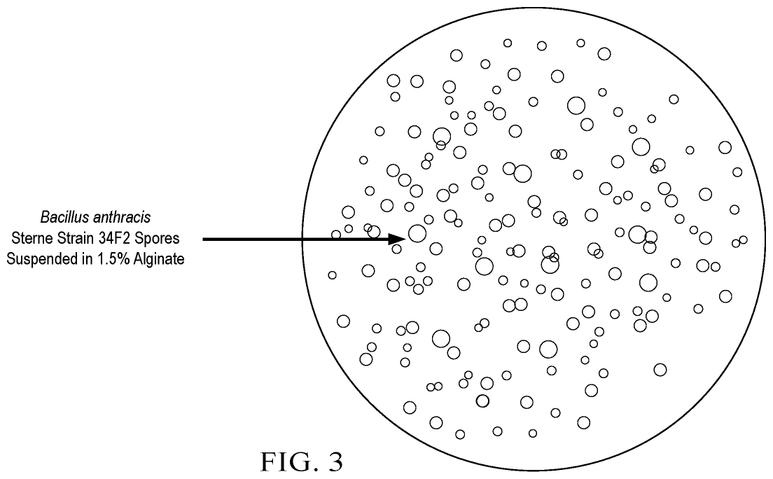

Microencapsulation of B. anthracis Sterne strain 34F2 spores. Multiple microcapsule vaccine formulations were prepared for the experiments in this study: (i) uncoated microcapsules containing 10⁹ spores/ml (FIG. 3), (ii) coated microcapsules with a PLL shell, and (iii) coated microcapsules with a PLL and VpB protein shell (Patent Application No. PCT/US2021/036834) also containing 10⁹ spores/ml.

Microcapsules were prepared similar to previous studies.³¹ Sodium alginate (NovaMatrix, Sandvika, Norway) was dissolved in MOPS buffer to a concentration of 1.5% (w/v) alginate. The skilled artisan will understand that the concentration (w/v) of alginate can be selected, such as 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% w/v. To make capsules, Sterne spores were suspended in MOPS buffer, sterile water, 2.5 mM D-alanine or 5 mM D-alanine and then mixed with 5 ml of 1.5% (w/v) alginate solution. Microcapsules were formed using a Nisco Encapsulator VARV1 unit (Nisco Engineering AG, Zurich, Switzerland). The spore+alginate solution was extruded through a 170 m nozzle, released directly into cross-linking solution (100 mM CaCl₂, 10 mM MOPS) and stirred for 30 minutes. For the uncoated formulation, capsules were thoroughly washed with MOPS, then collected and stored in MOPS until use. For the coated formulation, microcapsules were thoroughly washed with MOPS, and then coated with PLL or the protein shell by stirring for 30 minutes in 0.05% PLL and VpB in cross-linking solution (Patent Application No. PCT/US2021/036834). After another washing with MOPS, the capsules received an outer shell of 0.03% (w/v) alginate by mixing for 5 minutes.

Characterization of microcapsules in simulated gastrointestinal environments. Microcapsule morphology and bacterial presence within the alginate capsules were visualized with brightfield microscopy. Capsule responses, without and with a protein shell to simulated gastrointestinal fluids (GI fluids) were examined by suspending an aliquot of each capsule formulation in separate vials of the GI fluids. Vials were placed on a tube rocker at 37° C. and samples were collected at 30 and 90 minutes for imaging on an Olympus CKX41 microscope. Capsule diameters were measured in ImageJ.

White-tailed deer immunizations. Female white-tailed deer were donated by a private deer breeder for use in the experiment. Deer were housed at the deer breeder's facility for the entirety of the experiment and were randomly distributed into four groups of five deer each. All animal care and experimental procedures were performed in compliance with all Texas A&M University Institutional Animal Care and Use Committee regulations.

White-tailed deer were subcutaneously hand-injected, intramuscularly dart-injected or orally inoculated by needle-free syringe with one of the tested vaccines: (i) Sterne Vaccine, (ii) 1.2 ml of uncoated capsules suspended in MOPS, (iii) 1.2 ml of capsules coated with PLL, (iv) 1.2 ml of capsules coated with PLL and VpB, (v) 6 ml of uncoated capsules suspended in MOPS, (vi) 6 ml of capsules coated with PLL and VpB suspended in MOPS (Table 1). Sterne spore doses for each vaccine are detailed in Table 1. All deer received a prime vaccination dose and a booster dose of their respective vaccines two weeks later. Antibody responses were evaluated in blood samples that were collected prior to vaccination, every 10 to 14 days after vaccination for eight weeks, then approximately every 28 days for another 3-5 months.

TABLE 1
Vaccination groups to assess the efficacy of microencapsulated
Sterne spores as an oral vaccine.

| | Group | Inoculation | Spores/ | Blood Collection |
|---|---|---|---|---|
| Route | (n = 5) | Volume | Deer | (days post-vaccination) |
| SC | Sterne Vaccine | 1 ml | 5 × 10⁶ | 0, 14, 28, 42, 56, |
| | | | | 84, 112, 140 |
| IM | Sterne Vaccine | 1 ml | 5 × 10⁶ | 0, 14, 28, 42, 56, |
| | | | | 84, 112, 140 |
| Oral | Uncoated Capsules - | 1.2 ml solid capsules + | 10⁹ | 0, 14, 28, 42, 56, |
| | Small Dose | 3-4 ml MOPS | | 84, 112, 140 |
| | Uncoated Capsules - | 6 ml solid capsules + | 10¹⁰ | 0, 14, 28, 42, 56, |
| | Large Dose | 12-14 ml MOPS | | 84, 112, 140 |
| | PLL Coated | 1.2 ml solid capsules + | 10⁹ | 0, 14, 28, 42, 56, |
| | Capsules | 3-4 ml MOPS | | 84, 112, 140 |
| | PLL/VpB Coated | 1.2 ml solid capsules + | 10⁹ | 0, 14, 28, 42, 56, |
| | Capsules | 3-4 ml MOPS | | 84, 112, 140 |
| | PLL/VpB Coated | 6 ml solid capsules + | 10¹⁰ | 0, 14, 28, 42, 56, |
| | Capsules - | 12-14 ml MOPS | | 84, 112, 140 |
| | Large Dose | | | |

SC = subcutaneous,
IM = intramuscular by remote delivered dart,
Sterne Vaccine = B. anthracis Sterne strain 34F2 spores in saponin,
Uncoated Capsules = Microcapsules containing B. anthracis Sterne strain 34F2 spores without any coating,
Coated Capsules = Microcapsules containing B. anthracis Sterne strain 34F2 spores coated with a stabilizing protein shell

Detection of anthrax-specific antibody levels. Anthrax specific antibody levels were measured by ELISA as described previously.¹² High binding ELISA plates were coated with 100 ng per well of anthrax protective antigen (List Biological Laboratories Inc., Campbell, CA, USA) in carbonate buffer, pH 9.6 and incubated at 37° C. for 1 hour, then overnight at 4° C. The plates were washed 3-5 times with phosphate buffered saline containing 0.5% Tween 20 (PBST). This washing step was repeated between each of the following steps. Next, the plates were blocked for 1 hour at 37° C. with 100 μl per well of 1% (w/v) bovine serum albumin in PBST (1% BSA). Serial dilutions of all serum samples were prepared in 1% BSA, loaded 100 μl per well and incubated for 1 hour at 37° C. The secondary antibody, Anti-Deer IgG (H+L) (SeraCare, Milford, MA, USA), was diluted 1:500 in 1% BSA and loaded 100 μl to a well with a 1 hour incubation at 37° C. TMB/E Substrate (Sigma-Aldrich, St. Louis, MO, USA) was added to each well and the reaction was stopped after 12 minutes with the addition of 100 μl of 0.5 M H₂SO₄. The optical density of all wells was read on a Tecan Infinite F50 Plate Reader at 450 nm. Samples (n=5) from each time point, at each dilution were run in duplicate and are reported as average absorbance values for a single dilution for all vaccination groups at each time point.

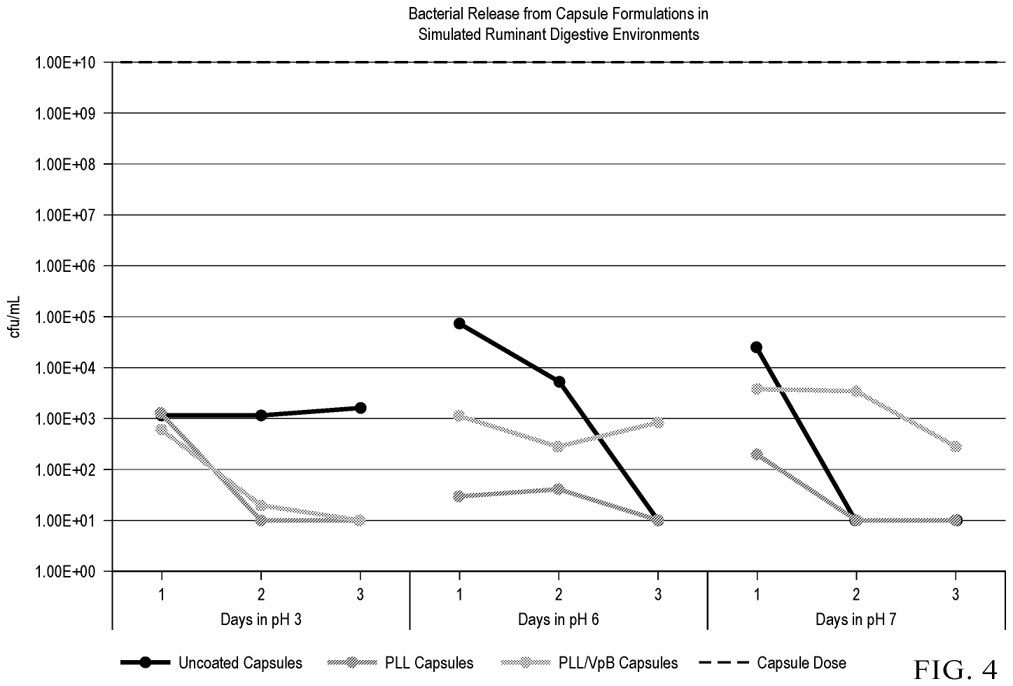

FIG. 4 is a graph that shows the release patterns of different microcapsule formulations with and without the protein shell in simulated ruminant digestive environments. Capsules were loaded with 10¹⁰ cfu/mL of Sterne spores and exposed to a simulated gastric environment to represent the abomasum (0.2% (w/v) NaCl, pH 3), and a simulated rumen, reticulum, omasum, and small intestine environment (0.68% (w/v) KH₂PO₄, pH 6 and 7) for 3 days.

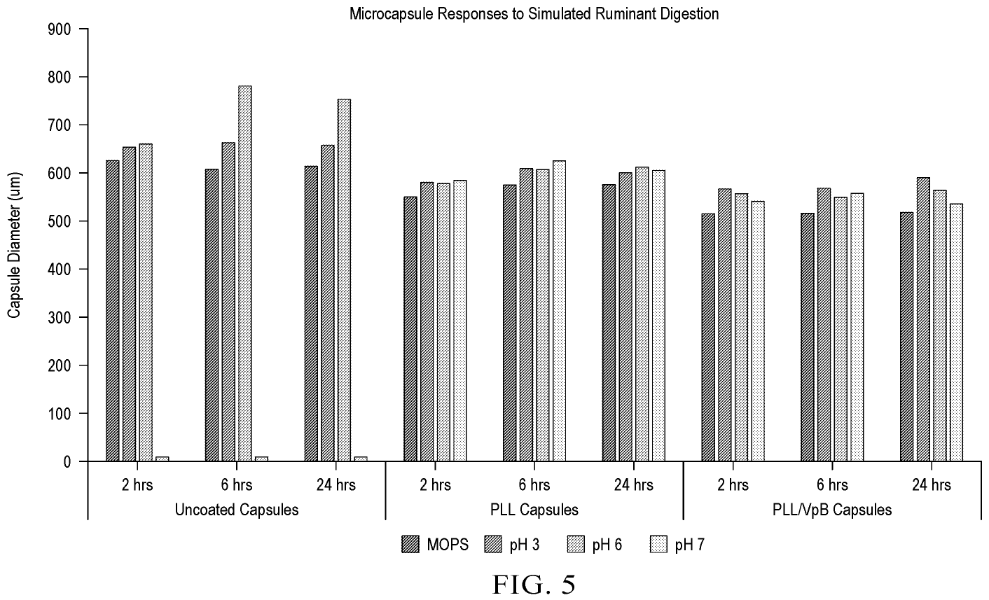

FIG. 5 is a graph that shows microcapsule diameter changes for different microcapsule formulations to simulated ruminant digestive environments for the estimated time that capsules would spend in each digestive organ. Uncoated capsules, PLL capsules and PLL/VpB capsules were suspended in simulated abomasum (0.2% (w/v) NaCl, pH 2 and pH 5) and simulated rumen, reticulum, omasum, and small intestine environments (0.68% (w/v) KH₂PO₄, pH 7 and 8) for 2 hours, 6 hours and 24 hours at 37° C. with shaking. Microcapsule samples were also suspended in MOPS buffer (10 mM MOPS, 0.85% NaCl) as a negative control for encapsulated vaccine storage conditions. The capsule diameters after exposure to simulated gastrointestinal fluids were observed in brightfield and measured in ImageJ. Data is reported as the average capsule diameter for the group in m.

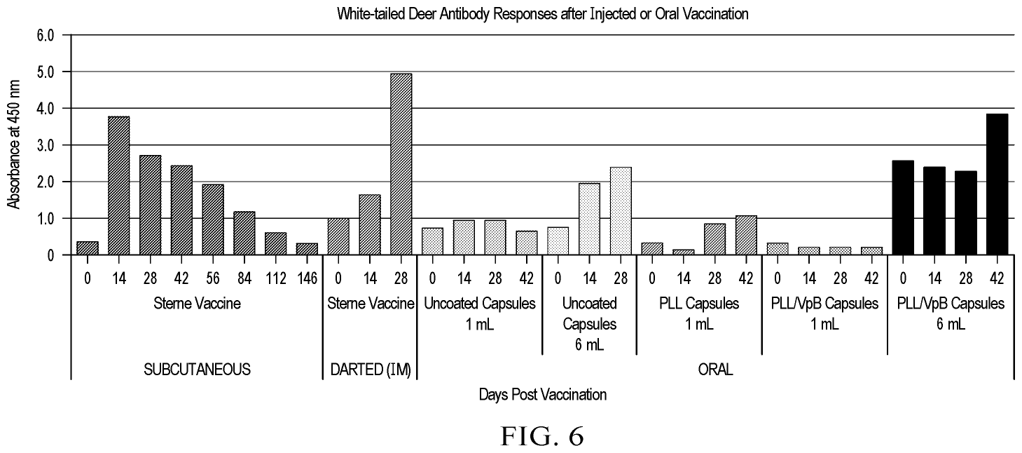

FIG. 6 is a graph that shows IgG responses in white-tailed deer following subcutaneous and intramuscular vaccination with the Sterne vaccine or oral vaccination with uncoated capsules, PLL capsules and PLL/VpB capsules at different doses. White-tailed deer were either subcutaneously injected or intramuscularly injected by remote delivered dart with the commercial Sterne Vaccine containing 10⁶ unencapsulated B. anthracis Sterne strain 34F2 spores in saponin, or they were orally vaccinated with 10⁹ encapsulated Sterne spores in 1.2 mL of uncoated capsules, 10¹⁰ encapsulated Sterne spores in 6 mL of uncoated capsules, 10⁹ encapsulated Sterne spores in 1 mL of PLL capsules, 10⁹ encapsulated Sterne spores in 1.2 mL of PLL/VpB capsules or 10¹⁰ encapsulated Sterne spores in 6 mL of PLL/VpB capsules. Serum samples were collected at 0, 14, 28, 42, 56, 84, 112 and 146-days post vaccination for the subcutaneous group, and 0, 14, 28 and 42-days for the oral groups. All serum samples were analyzed by ELISA.

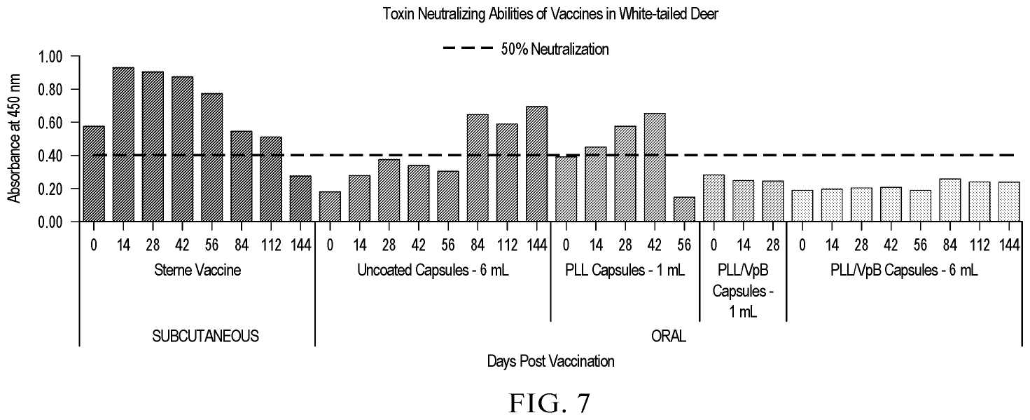

FIG. 7 is a graph that shows in vitro toxin neutralizing abilities of antibodies from subcutaneous administered Sterne Vaccine and orally administered uncoated capsules, PLL capsules and PLL/VpB capsules at different doses in white-tailed deer. Serum was collected from white-tailed deer at 0, 14, 28, 42, 56, 84, 112 and 144-days post vaccination following subcutaneous vaccination with 10⁶ unencapsulated B. anthracis Sterne strain 34F2 spores, and oral vaccination with 10¹⁰ encapsulated Sterne spores in 6 mL of uncoated capsules, 10⁹ encapsulated Sterne spores in 1.2 mL of PLL capsules, 10⁹ encapsulated Sterne spores in 1.2 mL of PLL/VpB capsules and 10¹⁰ encapsulated Sterne spores in 6 mL of PLL/VpB capsules. Diluted serum samples were pre-incubated with LeTx then added to J774A.1 cells and the resulting cell viability was assessed with MTT dye. Data presented here represents the average absorbance at 595 n.

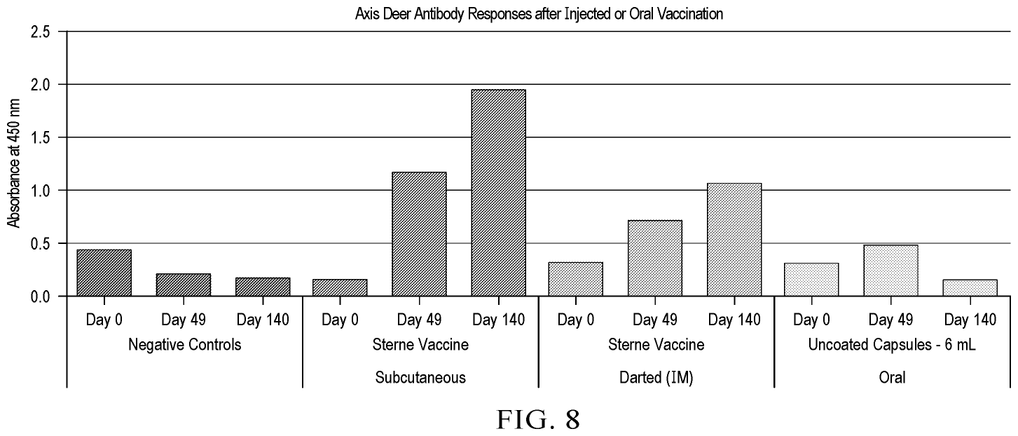

FIG. 8 is a graph that shows IgG responses in axis deer following subcutaneous and intramuscular vaccination with the Sterne vaccine or oral vaccination with uncoated capsules in axis deer. Axis deer were either subcutaneously injected or intramuscularly injected by remote delivered dart with the commercial Sterne Vaccine containing 10⁶ unencapsulated B. anthracis Sterne strain 34F2 spores in saponin, or they were orally vaccinated with 10¹⁰ encapsulated Sterne spores in 6 mL of uncoated capsules. Serum samples were collected at 0, 49, and 140-days post vaccination. All serum samples were analyzed by ELISA.

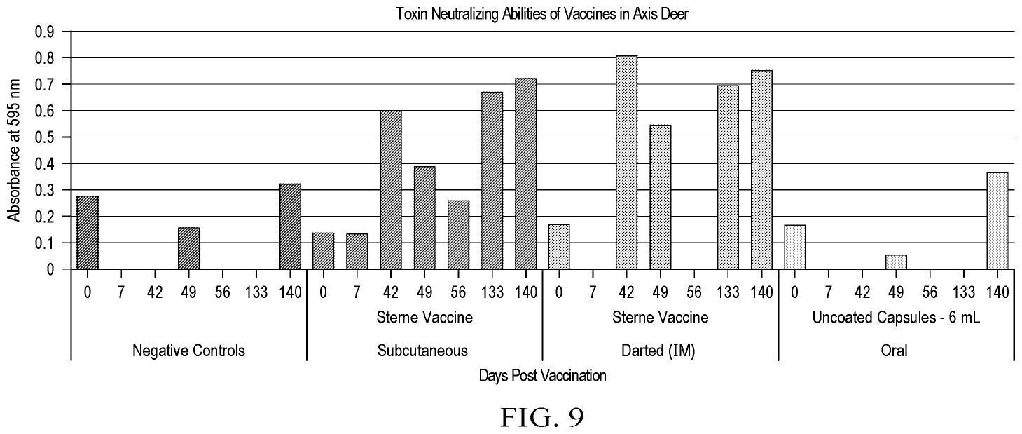

FIG. 9 is a graph that shows in vitro toxin neutralizing abilities of antibodies from subcutaneous or intramuscularly injected Sterne Vaccine and 6 mL of orally administered uncoated capsules. Serum was collected from axis deer at various time points including 0, 7, 42, 49, 56, 133 and 140-days post vaccination following subcutaneous vaccination with 10⁶ unencapsulated B. anthracis Sterne strain 34F2 spores, intramuscular vaccination with 10⁶ unencapsulated B. anthracis Sterne strain 34F2 spores, or oral vaccination with 10¹⁰ encapsulated Sterne spores in 6 mL of uncoated capsules. Diluted serum samples were pre-incubated with LeTx then added to J774A.1 cells and the resulting cell viability was assessed with MTT dye. Data presented here represents the average absorbance at 595 nm.

In one embodiment, the present invention includes an immunization against Bacillus anthracis comprising, consisting essentially of, or consisting of: B. anthracis Sterne strain 34F2 spores suspended in an alginate in an amount sufficient to protect an animal or human from a lethal dose of anthrax. In one aspect, the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the oral immunization further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5. 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

In another embodiment, the present invention includes a vaccine comprising, consisting essentially of, or consisting of: B. anthracis Sterne strain 34F2 spores suspended in an alginate, wherein the spores are provided in an amount sufficient to protect an animal or human from a lethal dose of anthrax. In one aspect, the immunization is for oral administration further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the vaccine further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 02.5, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

In another embodiment, the present invention includes a method for prophylaxis, amelioration of symptoms, or any combinations thereof against Bacillus anthracis in a human or animal subject comprising, consisting essentially of, or consisting of, the steps of: identifying the human or animal subject in need of the prophylaxis, amelioration of symptoms, or any combinations thereof against Bacillus anthracis; and administering a therapeutically effective amount of an attenuated immunization against Bacillus anthracis comprising: B. anthracis Sterne strain 34F2 spores suspended in an alginate, wherein the immunization is provided in an amount sufficient to protect an animal or human from a lethal dose of anthrax. In one aspect, the immunization is an oral immunization and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the B. anthracis Sterne strain 34F2 spores survive exposure to gastric conditions. In another aspect, the immunization further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 2, 2.5, 3, 4, 5, 6, 7, 8, 9, 10% weigh to volume (w/v).

In another embodiment, the present invention includes a method of making an attenuated vaccine against anthrax (Bacillus anthracis) comprising, consisting essentially of, or consisting of: suspending a B. anthracis Sterne strain 34F2 spores in alginate, wherein the alginate neutralizes positively charged amino acids, wherein the amount of the vaccine is sufficient to protect an animal or human from a lethal dose of anthrax. In one aspect, the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension. In another aspect, the vaccine further comprises at least one of a pharmaceutically acceptable carrier, bait, feed, pellets, tablets, lozenges, gelcaps, or capsules. In another aspect, the capsule comprising the Sterne strain 34F2 spores is not coated. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.25, 0.3, 0.4, 0.5, 0.75, 1, 2, 2.5, 3, 4, 5, 6, 7, 8, 9, 10 or more mM. In another aspect, the alginate is at 0.1, 0.2, 0.25, 0.3, 0.4, 0.5, 0.75, 1, 2, 2.5, 3, 4, 5, 6, 7, 8, 9, 10% weigh to volume (w/v).

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Hugh-Jones, M. E. & de Vos, V. Anthrax and wildlife. Rev. Sci. Tech. 21, 359-83 (2002).
2. Bacillus anthracis and Anthrax. Bacillus anthracis and Anthrax (Wiley-Blackwell, 2011). doi:10.1002/9780470891193
3. Carlson, C. J. et al. The global distribution of Bacillus anthracis and associated anthrax risk to humans, livestock and wildlife. Nat. Microbiol. 4, 1337-1343 (2019).
4. Sterne, M. Distribution and economic importance of anthrax. Fed Proc 26, 1493-1495 (1967).
5. Kracalik, I. et al. Changing livestock vaccination policy alters the epidemiology of human anthrax, Georgia, 2000-2013. Vaccine 35, 6283-6289 (2017).
6. Scorpio, A., Blank, T. E., Day, W. A. & Chabot, D. J. Anthrax vaccines: Pasteur to the present. Cellular and Molecular Life Sciences 63, 2237-2248 (2006).
7. Anthrax in Humans and Animals. in (ed. Turnbull, P.) (World Health Organization and International Office of Epizootics, 2008).
8. Brossier, F., Mock, M. & Sirard, J. C. Antigen delivery by attenuated Bacillus anthracis: New prospects in veterinary vaccines. J. Appl. Microbiol. 87, 298-302 (1999).
9. Hugh-Jones, M. & Blackburn, J. The ecology of Bacillus anthracis. Mol. Aspects Med. 30, 356-367 (2009).
10. Fasanella, A., Galante, D., Garofolo, G. & Jones, M. H. Anthrax undervalued zoonosis. Vet Microbiol 140, 318-331 (2010).
11. Hudson, M. J. et al. Bacillus anthracis: Balancing innocent research with dual-use potential. International Journal of Medical Microbiology 298, 345-364 (2008).
12. Benn Felix, J., Chaki, S. P., Ficht, T. A., Rice-Ficht, A. C. & Cook, W. Bacillus anthracis Sterne Strain 34F2 Vaccine Antibody Dose Response by Subcutaneous and Oral Administration. Poult Fish Wildl Sci 7, 206 (2019).
13. Aloni-Grinstein, R. et al. Oral Spore Vaccine Based on Live Attenuated Nontoxinogenic Bacillus anthracis Expressing Recombinant Mutant Protective Antigen. Infect. Immun. 73, 4043-4053 (2005).
14. Gorantala, J. et al. A plant based protective antigen [PA(dIV)] vaccine expressed in chloroplasts demonstrates protective immunity in mice against anthrax. Vaccine 29, 4521-4533 (2011).
15. Gorantala, J. et al. Generation of protective immune response against anthrax by oral immunization with protective antigen plant-based vaccine. J. Biotechnol. 176, 1-10 (2014).
16. Sim, B. K. L. et al. Protection against inhalation anthrax by immunization with Salmonella enterica serovar Typhi Ty21a stably producing protective antigen of Bacillus anthracis. npj Vaccines (2017). doi:10.1038/s41541-017-0018-4
17. Brossier, F., Levy, M. & Mock, M. Anthrax spores make an essential contribution to vaccine efficacy. Infect. Immun. 70, 661-664 (2002).
18. Choo, M.-K. et al. TLR sensing of bacterial spore-associated RNA triggers host immune responses with detrimental effects. J. Exp. Med. 214, 1297-1311 (2017).
19. Rice-Ficht, A. C., Arenas-Gamboa, A. M., Kahl-McDonagh, M. M. & Ficht, T. A. Polymeric particles in vaccine delivery. Current Opinion in Microbiology 13, 106-112 (2010).
20. Cook, M. T., Tzortzis, G., Charalampopoulos, D. & Khutoryanskiy, V. V. Microencapsulation of probiotics for gastrointestinal delivery. J. Control. Release 162, 56-67 (2012).
21. Simó, G., Fernández-Fernández, E., Vila-Crespo, J., Ruipdrez, V. & Rodriguez-Nogales, J. M. Research progress in coating techniques of alginate gel polymer for cell encapsulation. Carbohydrate Polymers (2017). doi:10.1016/j.carbpol.2017.04.013
22. Gombotz, W. R. & Wee, S. F. Protein release from alginate matrices. Advanced Drug Delivery Reviews 64, 194-205 (2012).
23. Sundar, S., Kundu, J. & Kundu, S. C. Biopolymeric nanoparticles. Sci. Technol. Adv. Mater. 11, 014104 (2010).
24. De, S. & Robinson, D. Polymer relationships during preparation of chitosan-alginate and poly-l-lysine-alginate nanospheres. J. Control. Release 89, 101-112 (2003).
25. Martin, M. J., Lara-Villoslada, F., Ruiz, M. A. & Morales, M. E. Microencapsulation of bacteria: A review of different technologies and their impact on the probiotic effects. Innov. Food Sci. Emerg. Technol. 27, 15-25 (2015).
26. Thu, et al. Alginate polycation microcapsules: I. Interaction between alginate and polycation. Biomaterials 17, 1031-1040 (1996).
27. Herbert Waite, J. & Rice-Ficht, A. C. Eggshell precursor proteins of Fasciola hepatica, II. Microheterogeneity in vitelline protein B. Mol. Biochem. Parasitol. 54, 143-151 (1992).
28. Rice-Ficht, A. C., Dusek, K. A., John Kochevar, G. & Herbert Waite, J. Eggshell precursor proteins of Fasciola hepatica, I. Structure and expression of vitelline protein B. Mol. Biochem. Parasitol. 54, 129-141 (1992).
29. Arenas-Gamboa, et al. Oral vaccination with microencapsuled Strain 19 vaccine confers enhanced protection against Brucella abortus strain 2308 challenge in Red deer (Cervus elaphus elaphus). J. Wildl. Dis. 45, 1021-1029 (2009).
30. Arenas-Gamboa, A. M., Ficht, T. A., Kahl-McDonagh, M. M., Gomez, G. & Rice-Ficht, A. C. The Brucella abortus S19 ΔvjbR live vaccine candidate is safer than S19 and confers protection against wild-type challenge in BALB/c mice when delivered in a sustained-release vehicle. Infect. Immun. 77, 877-884 (2009).
31. Arenas-Gamboa, A. M., Ficht, T. A., Kahl-McDonagh, M. M. & Rice-Ficht, A. C. Immunization with a single dose of a microencapsulated Brucella melitensis mutant enhances protection against wild-type challenge. Infect. Immun. 76, 2448-2455 (2008).
32. Arenas-Gamboa, et al. Enhanced Immune Response of Red Deer (Cervus elaphus) to Live RB51 Vaccine Strain Using Composite Microspheres. 45, 165-173 (2009).
33. Cook, M. T., Tzortzis, G., Charalampopoulos, D. & Khutoryanskiy, V. V. Production and evaluation of dry alginate-chitosan microcapsules as an enteric delivery vehicle for probiotic bacteria. Biomacromolecules 12, 2834-2840 (2011).
34. Meeusen, E. N. T., Walker, J., Peters, A., Pastoret, P. P. & Jungersen, G. Current status of veterinary vaccines. Clin. Microbiol. Rev. 20, 489-510 (2007).
35. CDC & Ncird. U.S. Vaccines: Table 1 and Table 2.
36. Vela Ramirez, J. E., Sharpe, L. A. & Peppas, N. A. Current state and challenges in developing oral vaccines. Advanced Drug Delivery Reviews (2017). doi:10.1016/j.addr.2017.04.008
37. Giserod, O., Sannes, A. & Skjik-Brok, G. Microcapsules of alginate-chitosan. II. A study of capsule stability and permeability. Biomaterials 20, 773-783 (1999).
38. Thu, B. et al. Alginate polycation microcapsules: II. Some functional properties. Biomaterials 17, 1069-1079 (1996).
39. Kuo, C. K. & Ma, P. X. Maintaining dimensions and mechanical properties of ionically crosslinked alginate hydrogel scaffolds in vitro. J. Biomed. Mater. Res.—Part A (2008). doi:10.1002/jbm.a.31375
40. Chuang, J. J. et al. Effects of pH on the Shape of Alginate Particles and Its Release Behavior. Int. J. Polym. Sci. 2017, (2017).
41. Rasel, M. A. T. & Hasan, M. Formulation and evaluation of floating alginate beads of diclofenac sodium. Dhaka Univ. J. Pharm. Sci. (2012). doi:10.3329/dujps.vllil.12484
42. Iyer, C. & Kailasapathy, K. Effect of Co-encapsulation of Probiotics with Prebiotics on Increasing the Viability of Encapsulated Bacteria under In Vitro Acidic and Bile Salt Conditions and in Yogurt. J. Food Sci. 70, M18-M23 (2005).
43. Li, X. Y. et al. Preparation of alginate coated chitosan microparticles for vaccine delivery. BMC Biotechnol. 8, 1-11 (2008).
44. Praepanitchai, O. A., Noomhorm, A., Anal, A. K. & Potes, M. E. Survival and Behavior of Encapsulated Probiotics (Lactobacillus plantarum) in Calcium-Alginate-Soy Protein Isolate-Based Hydrogel Beads in Different Processing Conditions (pH and Temperature) and in Pasteurized Mango Juice. Biomed Res. Int. 2019, (2019).
45. Storni, T., Kindig, T. M., Senti, G. & Johansen, P. Immunity in response to particulate antigen-delivery systems. Advanced Drug Delivery Reviews (2005). doi:10.1016/j.addr.2004.09.008
46. Csaba, N. et al. Trimethyl chitosan nanoparticles encapsulated protective antigen Protects the mice against anthrax. Front. Immunol. (2018). doi:10.3389/fimmu.2018.00562
47. Shakya, K. P., Hugh-Jones, M. E. & Elzer, P. H. Evaluation of immune response to orally administered Sterne strain 34F2 anthrax vaccine. Vaccine 25, 5374-5377 (2007).
48. Hanson, J. F., Taft, S. C. & Weiss, A. A. Neutralizing antibodies and persistence of immunity following anthrax vaccination. Clin. Vaccine Immunol. 13, 208-213 (2006).
49. Reuveny, S. et al. Search for correlates of protective immunity conferred by anthrax vaccine. Infect. Immun. 69, 2888-2893 (2001).
50. Welkos, S. L., Keener, T. J. & Gibbs, P. H. Differences in Susceptibility of Inbred Mice to Bacillus-Anthracis. Infect. Immun. 51, 795-800 (1986).
51. Jang, S.-F. et al. Size discrimination in rat and mouse gastric emptying. Biopharm. Drug Dispos. 34, 107-124 (2013).
52. McGhee, J. R. et al. The mucosal immune system: from fundamental concepts to vaccine development. Vaccine 10, 75-88 (1992).
53. Vandamme, T. F. & Ellis, K. J. Issues and challenges in developing ruminal drug delivery systems. Adv. Drug Deliv. Rev. 56, 1415-1436 (2004).
54. Xu, Y., Liang, X., Chen, Y., Koehler, T. M. & Hook, M. Identification and biochemical characterization of two novel collagen binding MSCRAMMs of Bacillus anthracis. J Biol Chem 279, 51760-51768 (2004).
55. Russell, B. H., Vasan, R., Keene, D. R. & Xu, Y. Bacillus anthracis internalization by human fibroblasts and epithelial cells. Cell. Microbiol. 9, 1262-1274 (2007).
56. Russell, B. H., Vasan, R., Keene, D. R., Koehler, T. M. & Xu, Y. Potential dissemination of Bacillus anthracis utilizing human lung epithelial cells. Cell. Microbiol. 10, 945-957 (2008).
57. Jenkins, S. A. & Xu, Y. Characterization of Bacillus anthracis Persistence In Vivo. PLoS One 8, 1-9 (2013).
58. Basu, S. et al. Role of Bacillus anthracis spore structures in macrophage cytokine responses. Infect. Immun. 75, 2351-2358 (2007).
59. Parish, J., Rivera, J. & Boland, H. Understanding the ruminant animal digestive system. 1-5 (2009).

1. An immunization against Bacillus anthracis comprising:
B. anthracis Sterne strain 34F2 spores suspended in an alginate in an amount sufficient to protect an animal or human from a lethal dose of anthrax.

2. The immunization of claim 1, wherein the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension.

3. The immunization of claim 1, wherein the B. anthracis Sterne strain 34F2 spores survive exposure to gastric conditions.

4. The immunization of claim 1, wherein the oral immunization further comprises at least one of a pharmaceutically acceptable carrier, bait, feed, pellets, tablets, lozenges, gelcaps, or capsules; or the capsule comprising the Sterne strain 34F2 spores is not coated.

5. (canceled)

6. The immunization of claim 1, wherein the alginate further comprises at least one of:
an amount of D-alanine sufficient to prevent germination of the B. anthracis Sterne strain 34F2 spores,
an amount of D-alanine at an amount of 0.1, 0.2, 0.25, 0.3, 0.4, 0.5. 0.75, 1, 2, 2.5, 3, 4, 5, 6, 7, 8, 9, 10 or more mM; or
the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

7. (canceled)

8. (canceled)

9. A vaccine comprising:
B. anthracis Sterne strain 34F2 spores suspended, coated with, or both, in an alginate, wherein the spores are provided in an amount sufficient to protect an animal or human from a lethal dose of anthrax.

10. The vaccine of claim 9, wherein the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension.

11. The vaccine of claim 9, wherein the B. anthracis Sterne strain 34F2 spores survive exposure to gastric conditions: or the capsule comprising the Sterne strain 34F2 spores is not coated.

12. The vaccine of claim 9, wherein the vaccine further comprises at least one of a pharmaceutically acceptable carrier, bait, feed, pellets, tablets, lozenges, gelcaps, or capsules.

13. (canceled)

14. The vaccine of claim 9, wherein the alginate further at least one of:
an amount of D-alanine sufficient to prevent germination of the B. anthracis Sterne strain 34F2 spores,
an amount of D-alanine at an amount of 0.1, 0.2, 0.25, 0.3, 0.4, 0.5. 0.75, 1, 2, 2.5, 3, 4, 5, 6, 7, 8, 9, 10 or more mM: or
the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

15. (canceled)

16. (canceled)

17. A method for prophylaxis, amelioration of symptoms, or any combinations thereof against Bacillus anthracis in a human or animal subject comprising the steps of:
identifying the human or animal subject in need of the prophylaxis, amelioration of symptoms, or any combinations thereof against Bacillus anthracis; and
administering a therapeutically effective amount of an attenuated immunization against Bacillus anthracis comprising:
B. anthracis Sterne strain 34F2 spores suspended in an alginate, wherein the immunization is provided in an amount sufficient to protect an animal or human from a lethal dose of anthrax.

18. The method of claim 17, wherein the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension.

19. The method of claim 17, wherein the B. anthracis Sterne strain 34F2 spores survive exposure to gastric conditions: or the capsule comprising the Sterne strain 34F2 spores is not coated.

20. The method of claim 17, wherein the immunization further comprises at least one of a pharmaceutically acceptable carrier, bait, feed, pellets, tablets, lozenges, gelcaps, or capsules.

21. (canceled)

22. The method of claim 17, wherein the alginate further comprises at least one of:
an amount of D-alanine sufficient to prevent germination of the B. anthracis Sterne strain 34F2 spores,
an amount of D-alanine at an amount of 0.1, 0.2, 0.25, 0.3, 0.4, 0.5. 0.75, 1, 2, 2.5, 3, 4, 5, 6, 7, 8, 9, 10 or more mM: or
the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

23. (canceled)

24. (canceled)

25. A method of making an attenuated vaccine against anthrax (Bacillus anthracis) comprising:
suspending a B. anthracis Sterne strain 34F2 spores in alginate, wherein the alginate neutralizes positively charged amino acids, wherein the amount of the vaccine is sufficient to protect an animal or human from a lethal dose of anthrax.

26. The method of claim 25, wherein the immunization is oral and further comprises at least one of: an adjuvant, a delivery vehicle for at least one of a B. anthracis protective antigen, a B. anthracis edema factor, or a B. anthracis lethal factor, which are encapsulated separately, together, or in combination with the B. anthracis Sterne spores in suspension.

27. The method of claim 25, wherein the vaccine further comprises at least one of a pharmaceutically acceptable carrier, bait, feed, pellets, tablets, lozenges, gelcaps, or capsules.

28. The method of claim 27, wherein the capsule comprising the Sterne strain 34F2 spores is not coated: or the capsule comprising the Sterne strain 34F2 spores is not coated.

29. The method of claim 25, wherein the alginate further comprises at least one of:
an amount of D-alanine sufficient to prevent germination of the B. anthracis Sterne strain 34F2 spores,
an amount of D-alanine at an amount of 0.1, 0.2, 0.25, 0.3, 0.4, 0.5. 0.75, 1, 2, 2.5, 3, 4, 5, 6, 7, 8, 9, 10 or more mM: or
the alginate is at 0.1, 0.2, 0.25, 0.3, 0.4, 0.5, 0.75, 1, 1.25, 1.5, 1.75, 2, 2.25, 2.5, 2.75, 3, 4, 5, 6, 7, 8, 9, 10% weigh to volume (w/v).

30. (canceled)

31. (canceled)
